(19) United States
(12) Patent Application Publication
(10) Pub. No.: US 2002/0029063 A1
(43) Pub. Date: Mar. 7, 2002

(54) Prosthesis for abdominal surgery

(76) Inventor: Dietmar H. Wittmann, Nokomis, FL (US)

Correspondence Address: Irvin L. Groh, P.O., Gifford, Krass, Groh,, Sprinkle,Anderson & Citkowski, P.C., 280 N. Old Woodward Avenue, Suite 400, Birmingham, MI, 48009-5394, US

(21) Appl. No.: 09/947,070

(22) Filed: Sep. 5, 2001

Related U.S. Application Data
(60) Provisional application No. 60/230,202, filed on Sep. 5, 2000.

(51) Int. Cl. A61B 17/08

(52) U.S. Cl. 606/215

(57) ABSTRACT

A prosthesis for abdominal surgery and the method of using it, in which a two-sheet prosthetic is attached to the facia only at opposite sides of an incision. The sheets permit opening and reclosing of the incision and maintaining tension on the facia to bring the edges closer together with each opening and closing to allow for final fascia-to-fascia closure. The method of detoxifying the two-sheet facia prosthesis also is disclosed.

This application claims priority based on Provisional Patent Application No. 0/230,202 filed Sep. 5, 2000.

### FIELD OF THE INVENTION

This invention relates to abdominal surgery devices and the method of using and detoxifying such devices.

### BACKGROUND OF THE INVENTION

There are a number of abdominal surgery emergencies especially for trauma and infectious conditions, which cannot be solved with a single operation and multiple re-operations are required. In these conditions, intra-abdominal pressure is often increased and closing the abdomen forcefully leads to multi-organ failure and death. Traditionally, the surgeon was always closing the abdomen by suturing the fascia. He would be reoperating when clinically the need for a re-operation became obvious. This, however, caused a delay in diagnosis which consequently resulted in a high mortality in such conditions. The need to treat increased intra-abdominal pressure to prevent abdominal compartment syndrome and multi-organ dysfunction was traditionally neglected until very recently (1995). To treat increased intra-abdominal pressure, the abdominal cavity was initially left open which lead to bowel fistulae and high incisional hernias that were extremely difficult to manage.

To avoid the delay in diagnosis of postoperative intra-abdominal emergencies the conception of planned relaparotomy was designed. Temporary closure methods using retention sutures wires were employed and severe abdominal wall necrosis and necrotizing fasciitis were observed. Later other devices such as plastic meshes were introduced to act as fascial prostheses and cover abdominal content. Plastic meshes, however, need to be reopened and often replaced for abdominal re-entry. Reapproximation of the fasciae is rarely possible using prior art devices and high abdominal hernias develop in most cases.

As treatment progresses, most of the prior art devices cannot accommodate decreases in abdominal distention and have to be replaced by a similar member and resutured.

There is obviously a need for a method and fascial prosthesis for temporary bridging the fascial gap and permitting final fascial closure without leaving foreign material in situ at the same time.

More precisely a need exists for a simple, effective, improved method and device use for decompressing abdominal hypertension; for protecting exposed abdominal contents; for opening and closing incisions without tissue damage; for permitting final fascial closure without leaving a foreign body in place; and for averting infectious risks during the entire prosess.

### BRIEF SUMMARY OF THE INVENTION

The objects of the present invention are to provide a simple, effective, improved method and fascial prosthesis device for bridging fascial gaps, decompressing increased abdominal pressure, protecting abdominal contents, temporary closing the incision so that it can be subsequently opened and re-closed as needed, and finally closing the abdomen fascia to fascia without need for prosthetic material and preventing bacterial contamination during the decompression and reclosure period at the same time.

The device of the present invention basically comprises two flexible, trimmable sterile sheets. The first flexible, trimmable sheet has a relatively smooth bottom for covering the wound, and a top surface which will mate with or from a cohesive, releasable bond with the bottom surface of the second, flexible, trimmable sheet.

In the preferred embodiment, the kit consists of a sterile package containing two sterile rectangular sheets of plastic Velcro-like material, each about 20×40 cm. One sheet is characterized by having “hooks” (micromushrooms) on the bottom, while the other sheet has a top with “loops” to which the “hooks” attach when the two pieces are placed one upon the other with the “hooks” on the “loops”. The “hooks” are not hooks in the classical sense. They consist of micromushrooms which functionally act as hooks. Once thus joined the sheets can only be disconnected by lifting and separating one sheet from the other. They cannot be separated by pulling the sheets apart edge to edge.

In the methods of the present invention, one edge of the first sheet is attached with the top of the sheet with its mating surface with loops facing upward, and the relatively smooth bottom facing downward. The first sheet is sutured to the fascia and the free end is inserted between the opposite parietal peritoneum and the intentines so that the first sheet protects any exposed abdominal contents. The second flexible, trimmable sheet is then similarly sutured to the opposite fascia of the abdominal wound with the bottom-mating surface of micromushrooms facing downward. To temporarily close the wound a slight pulling is exerted on each of the two sheets to put the fascia under minimal tension, and the mating surfaces are brought together to close the incision. Intra-abdominal organs may be protected during the process by covering the mating surfaces of the second sheet with a towel, as long as the abdomen is open.

Subsequently, when the abdominal re-exploration is performed, the bonds between the mating surfaces are broken and the sheets folded back to open the previously closed incision. If the wound is to be again temporarily closed, the process is reversed taking care to insure that both abdominal wall fasciae are again under minimal tension so that they do not retract. If, when the wound is recluse the edges of the fasciae are closer together than originally, the sheets can be tailored to the proper size by trimming them with scissors to an equivalent size to the distance between the opposed fascial edges and the excess material removed. The opening and closing can be repeated until the wound is ready to be permanently closed at which time the remainders of the two sheets are removed and the fascia joined by a continuous suture. During the entire procedure that may last several days, the abdominal opening with the fascial prosthesis is protected by the self adhesive plastic sheet cover and negative pressure applied to hermetically seal the abdominal aperture.

The novel method of the present invention is cost effective as it better uses hospital resources and it reduces both mortality and morbidity by avoiding formation of bowel fistulae and high abdominal hernias.

It will be apparent to those skilled in the art that the present invention fulfills the above-stated objects and also provides other advantages.

### BRIEF DESCRIPTIONS OF THE DRAWINGS

FIG. 1 is a perspective view of a kit of the present invention;

FIG. 2 is a perspective view showing the present invention closing an abdominal incision;

FIG. 3 is a view taken along lines 3-3 in FIG. 2;

FIG. 4 is another view like FIG. 2 showing the device of the invention being trimmed with scissors to remove excess material;

FIG. 5 is a view like FIG. 2 showing the abdominal incision closed after trimming; and, FIG. 6 is a view similar to FIG. 3 showing the final stage of applying the wound shield.

### DESCRIPTION OF PREFERRED EMBODIMENT

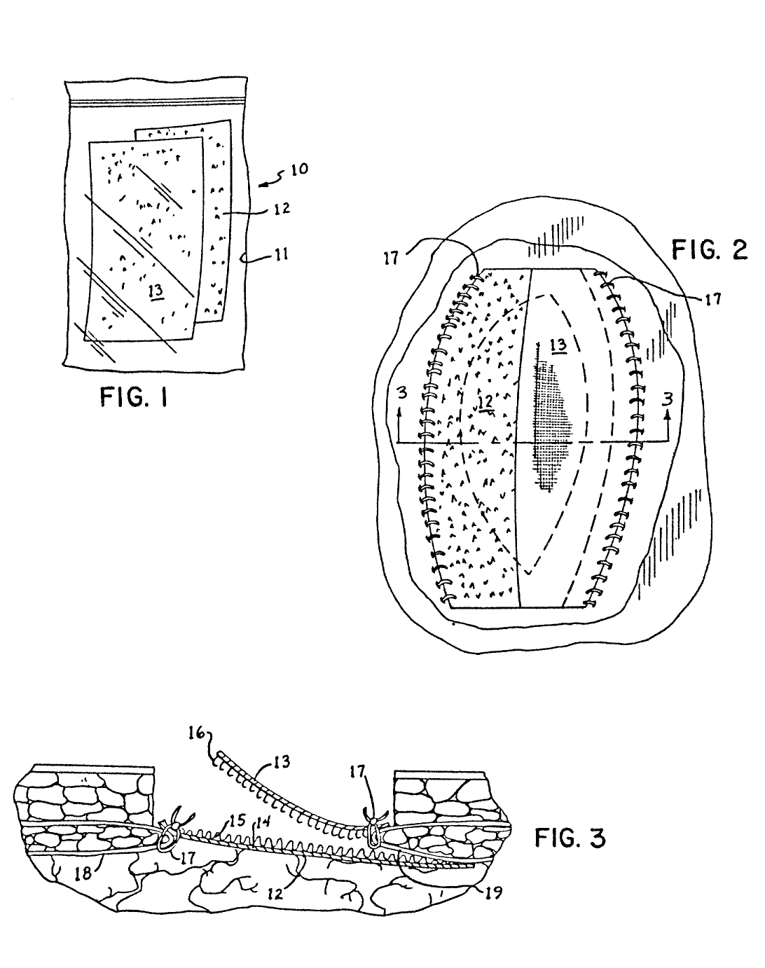

In the preferred embodiment of the invention shown in FIG. 1, the prosthesis or device 10 is stored in a sealed outer package 11 with a sterile interior which contains a sterile loop sheet 12 and a sterile micromushroom sheet 13.

As seen in FIG. 3, the sheet 12, has a smooth bottom 14, and a top 15, which is adapted to mate with the bottom 16, of the male sheet 13. Sheets 12 and 13 mate. The top 15, of the female sheet 12, is covered with “loops” and the bottom 16, of the male sheet 13, is provided with a multitude of “hook-like” protuberances (micromuchrooms) that mate 15 with the looped surface to releasable bond the sheets 12 and 13 together.

The sheets 12 and 13 should be made of a biocompatible, easily sterilized fabric which can be easily and securely sutured, and easily trimmed using conventional operating room instruments. Preferably, the sheets 12 and 13 are of a polyester material, and they are of contrasting colors to avoid confusion. The loop sheet 12 is made of a 20 polymer and the micromushroom sheet 13 is made of a polymer, polypropylene, polyurethane and polyacrylate.

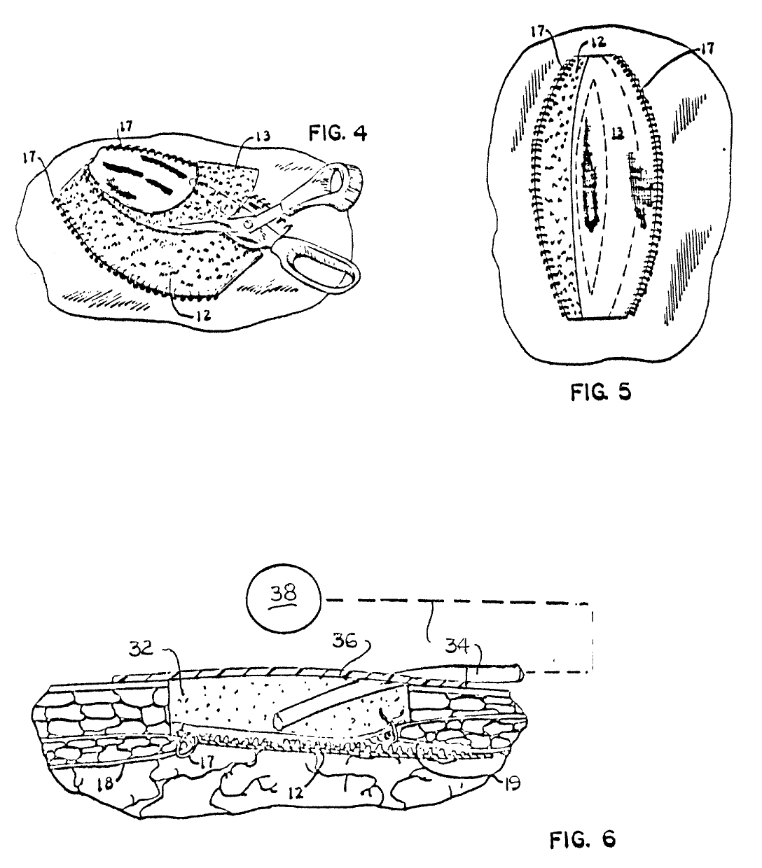

The preferred method of using the device or implant of the present invention will be described in conjunction with FIGS. 2 to 5.

When it is desired to close an incision, as for example at the end of a laparotomy, one edge of the loop sheet 12, is attached with a runring suture 17, to one fascia 18 with the top or loop side 15 up, i.e., so that the loop side does not contact bowel wall, omentum, or other intraperitoneal organs. The smooth biocompatible bottom of the loop sheet 12 protects the exposed abdominal contents and the free end of the loop sheet is inserted between the parietal peritoneum and the intestines at the opposite edge of the wound. One edge of the hook sheet 13, which is preferably of a contrasting color, is similarly sutured to the opposite fascia 19 so that the bottom or hooks side 16 will face toward the loop side 15 of sheet 12, which is covering the abdominal organs. Then the fascial edges are approximated by pulling the free edges of both of the sheets 12 and 13 toward each other to exert a minimal positive tension on the fascia and the cohesive surfaces are mated to close the incision and make a temporary abdominal closure.

In FIG. 3, the sheets 12 and 13 can be seen overlapped and bonded together by the mating of the micromushroom top 15 and bottom 16. When thus mated the sheets 12 and 13 cannot be separated except by lifting the male sheet 13 of the female sheet 12.

Gauze 32 is utilized to cover the hook sheet 12 and subcutaneous tissue up to the level of the skin. A suction drain tube 34 is imbedded into the gauze 32. Following this, a plastic drape 36 having an adhesive side is applied to the skin to cover the entire abdominal wall and the wound, leaving a tunnel for the drain tube. This seals the abdominal cavity and keeps it sterile. The area of the skin covered by the plastic drape 36 should cover a distance of at least 20 cm from any edge of the abdominal wound. This plastic drape seals off the abdominal cavity and a suction is applied to the drain tube 32 by a pump 38 to provide a sealing negative pressure and to collect abdominal fluid for measurement of protein losses and other factors for possible replacement.

The interval between two operations of a series of planned abdominal re-entries or staged abdominal repairs should not exceed thirty-six hours after the ending of the previous abdominal entry. It is important to definitively close the abdomen as early as possible when most of the peritoneal edema has disappeared. With every abdominal reentry the fascial edges should be pulled together to decrease the gap between the fascias.

The abdominal cavity can be finally closed once the problem within the abdominal cavity is solved. The sheets 12 and 13 are removed by taking out the running sutures. The hook sheet 13 is first removed from one side and then the loop sheet 12 from the other side. Subsequent to this, the fascia is closed by conventional suture technique.

The device 10 incorporating sheets 12 and 13 must be detoxified for use in abdominal surgery. For that purpose, the sheets 12 and 13 of hook and loop material are cut to a predetermined size to accommodate the largest of sizes of expected incisions and are measured for compliance. The sheets are easily trimmed to a smaller size at the time of surgery.

The sized sheets are immersed in an alcohol solution and remain immersed for an extended predetermined time. Upon removal from the alcohol solution the sheets are drained and rinsed with purified water.

The components subsequently are laid out in an orderly pattern in a room or hooded area and subjected to laminar airflow until the components are completely dry.

The dried components are packaged in an internally sterile package such as a transparent plastic bag for storage until use.

1. The method of making a sterile prosthesis of plastic material for temporarily closing a abdominal incision, comprising:
forming a first prosthetic member of plastic material
forming a second prosthetic member of plastic material.
immersing said prosthetic members in alcohol for a predetermined period of time,
rinsing said prosthetic members in purified water, and
exposing said prosthetic members to air to dry.

2. The method of claim 1 wherein said members that are dried are sterilized and placed in a package within a clean packaging area for storage until use.

3. The method of claim 1 wherein said members are placed in a selected area and are subjected to laminar airflow to be dried.

4. The method of claim 3 wherein said selected area of laminar airflow occurs under a hood.

5. The method of claim 2 wherein said package into which said members are placed is internally sterile.

6. The method of making a device for use in abdominal surgery in which the device contains a pair of flexible fastener elements of plastic sheet material, said method comprising:
forming said fastener elements to a selected size,
immersing said elements in alcohol for a selected period of time,
draining said alcohol from said elements,
rinsing said elements in purified water,
drying said elements in a selected area, and
placing said elements in a container.

7. The method of claim 6 wherein said area for drying is subjected to airflow.

8. The method of claim 6 wherein said container is internally sterile.

9. The method of temporarily closing an abdominal incision to permit repeated opening and closing comprising:
attaching one edge of a flexible prosthetic sheet to the fascia at a first edge of an incision,
said first and second prosthetic sheets having complementary closure surfaces facing each other,
closing said fastening means with a positive tension on each of the edges of the fascia,
covering the space between the edges of the fascia and above the closed fastening means with a gauze,
imbedding a drain tube under continuous negative pressure in said gauze,
covering the area of the incision with a sheet of self-adhering plastic material having a border larger than the area covered by said gauze to engage the skin adjacent to the incision.

10. The method of claim 9 wherein reopening and closing of said prosthetic sheets includes the step of applying tension to opposed edges of the fascia prior to reclosing said prosthetic sheets.

11. The method of claim 10 and further comprising the step of trimming excess material from said prosthetic sheets at the time of reclosing said sheets.
